(19) United States
(12) Patent Application Publication
(10) Pub. No.: US 2024/0050613 A1
(43) Pub. Date: Feb. 15, 2024

(54) STERILIZING DEVICE

(71) Applicant: Shenzhen Shenlongke Technology Co., LTD, Shenzhen City (CN)

(72) Inventor: Gang JIANG, Shenzhen City (CN)

(21) Appl. No.: 17/965,919

(22) Filed: Oct. 14, 2022

(30) Foreign Application Priority Data
Aug. 31, 2022 (CN) 202222326856.0

(51) Int. Cl. A61L 2/26; A61L 2/08

(52) U.S. Cl. CPC A61L 2/26; A61L 2/08; A61L 2202/122; A61L 2202/121; A61L 2202/11

(57) ABSTRACT

Disclosed is a sterilizing device, which may include: a housing and a cover, the cover being configured to open or cover relative to the housing, and in a state that the cover is configured to cover relative to the housing, the housing and the cover jointly forming a storage space; a sterilizing lamp mounted on one of the housing or the cover, and configured to emit sterilizing light towards the storage space; and an optical reflector arranged on the other one of the housing or the cover, and opposite to the sterilizing lamp in the storage space.

### CROSS-REFERENCE TO RELATED APPLICATION

This application claims the benefit of Chinese Application No. 202222326856.0 filed Aug. 31, 2022, titled STERILIZING DEVICE, the entire contents of which are incorporated herein by reference.

### FIELD

The present disclosure relates to the field of sterilizing technologies, and more particularly, to a sterilizing device.

### BACKGROUND

Personal articles having high requirements for sanitary conditions, such as toothbrushes, tableware, dentures and razors, may breed bacteria and viruses after long-term use. Therefore, a sterilizing device for sterilizing these personal articles is proposed. At present, an ultraviolet sterilizing lamp is generally used in the sterilizing device for sterilizing.

Although the sterilizing device can effectively sterilize an object to be sterilized, a distribution area of the sterilizing lamp in the sterilizing device is limited, which may lead to the inability to sterilize the object in all directions.

### SUMMARY

The present disclosure aims to solve at least one of the technical problems in the prior art to some extent. Therefore, the present disclosure provides a sterilizing device capable of sterilizing an object to be sterilized from more directions.

A sterilizing device according to an aspect of the present disclosure may include a housing and a cover, wherein the cover is configured to open or cover relative to the housing, and in a state that the cover is configured to cover relative to the housing, the housing and the cover jointly form a storage space; a sterilizing lamp mounted in one of the housing or the cover, and configured to emit sterilizing light towards the storage space; and an optical reflector arranged on the other one of the housing or the cover, and opposite to the sterilizing lamp in the storage space.

The sterilizing device of the present disclosure has the following beneficial effect that the sterilizing device can sterilize an object to be sterilized from more directions.

In some embodiments, the sterilizing lamp is mounted at a bottom portion in the housing, and the optical reflector is arranged at a top portion in the cover.

In some embodiments, a plurality of sterilizing lamps are provided, and the plurality of sterilizing lamps are distributed at intervals at the bottom portion in the housing; and in the state that the cover is configured to cover relative to the housing, the optical reflector shields the plurality of the sterilizing lamps from a top-view perspective.

In some embodiments, the optical reflector is a reflecting mirror.

In some embodiments, the sterilizing lamp emits the sterilizing light towards the storage space through an optical refractor.

In some embodiments, the sterilizing device is also provided with a channel portion, and the channel portion communicates the storage space with an exterior of the sterilizing device.

In some embodiments, the cover is hinged with the housing, and the channel portion is formed in a hinged area of the cover and the housing.

In some embodiments, a detachable filtering member is arranged in the channel portion.

In some embodiments, a detachable fixing member is arranged in the channel portion, and the filtering member is fixed in the channel portion by the fixing member

In some embodiments, the fixing member is provided with a ventilation hole arranged at one end of the fixing member, wherein in a state that the fixing member is arranged in the channel portion, the ventilation hole is connected with the storage space; a ventilation groove arranged at the other end of the fixing member, wherein in the state that the fixing member is arranged in the channel portion, the ventilation groove is connected with the exterior of the sterilizing device; and an avoidance portion arranged between the ventilation hole and the ventilation groove, wherein in the state that the fixing member is arranged in the channel portion, the avoidance portion is arranged opposite to an inner wall of the channel portion and forms a mounting space for mounting the filtering member together with the inner wall.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a top view of an embodiment of a sterilizing device of the present disclosure;

FIG. 2 is a schematic diagram of the sterilizing device in FIG. 1 in an opened state;

FIG. 3 is a cross-sectional view along an A-A part in FIG. 1;

FIG. 4 is a schematic diagram of a propagation track of sterilizing light;

FIG. 5 is a perspective diagram of an embodiment of a housing of the sterilizing device of the present disclosure;

FIG. 6 is a perspective diagram of an embodiment of a cover of the sterilizing device of the present disclosure;

FIG. 7 is an enlarged view of a B part in FIG. 3; and

FIG. 8 is a perspective diagram of an embodiment of a fixing member in FIG. 3.

### REFERENCE NUMERALS

101 refers to housing; 102 refers to cover; 103 refers to sterilizing lamp; 104 refers to optical reflector; 105 refers to storage space; 106 refers to outer housing; 107 refers to inner housing; 108 refers to first substrate; 109 refers to first annular wall; 110 refers to second substrate; 111 refers to second annular wall; 112 refers to control box; 113 refers to control panel; 114 refers to battery; 115 refers to first mounting wall; 116 refers to power switch; 117 refers to charging interface; 118 refers to sealing plug; 119 refers to mounting hole; 120 refers to gasket; 121 refers to first arc-shaped portion; 122 refers to second arc-shaped portion; 123 refers to third arc-shaped portion; 124 refers to channel portion; 125 refers to first mounting structure; 126 refers to second mounting structure; 127 refers to mounting groove; 128 refers to second mounting wall; 129 refers to hinge shaft; 130 refers to hinge hole; 131 refers to hinged area; 132 refers to filtering member; 133 refers to gap portion; 134 refers to optical refractor; 135 refers to inner wall; 136 refers to supporting surface; 137 refers to fixing member; 138 refers to upper end portion; 139 refers to lower end portion; 140 refers to avoidance portion; 141 refers to ventilation hole; 142 refers to ventilation groove; 143 refers to sealing ring; and 200 refers to object to be sterilized.

### DETAILED DESCRIPTION

Embodiments of the present disclosure are described in detail hereinafter, examples of the embodiments are shown in the drawings, wherein the same or similar reference numerals throughout the drawings denote the same or similar elements or elements having the same or similar functions. The embodiments described hereinafter with reference to the drawings are examples, and are only intended to explain the present disclosure, but should not be understood as limiting the present disclosure.

In the description of the present disclosure, it should be understood that, when the description of orientation is involved, the orientations or positional relationships indicated by the terms such as “up”, “down”, “front”, “rear”, “left”, “right”, and the like, refer to the orientations or positional relationships shown in the drawings, which are only intended to facilitate describing the present disclosure and simplifying the description, and do not indicate or imply that the indicated devices or elements must have a specific orientation, be constructed and operated in a specific orientation, and therefore cannot be understood as a limitation of the present disclosure.

In the description of the present disclosure, the term “several” refers to being one or more, the term “multiple” refers to being more than two, and the terms such as “greater than”, “less than”, “more than” and the like are understood as not including this number, while the terms such as “above”, “below”, “within” and the like are understood as including this number. If there is the description of first and second, it is only for the purpose of distinguishing technical features, and should not be understood as indicating or implying relative importance or implying the number of indicated technical features or implying the order of indicated technical features.

In the description of the present disclosure, the terms such as “arrangement”, “installation”, “connection”, and the like should be understood in broad sense unless otherwise specified and defined. The specific meanings of the above terms in the present disclosure may be reasonably determined according to specific contents of the technical solutions by those skilled in the art.

In the description of the present disclosure, the same or similar technical features are provided with the same reference numerals, and are only distinguished when necessary.

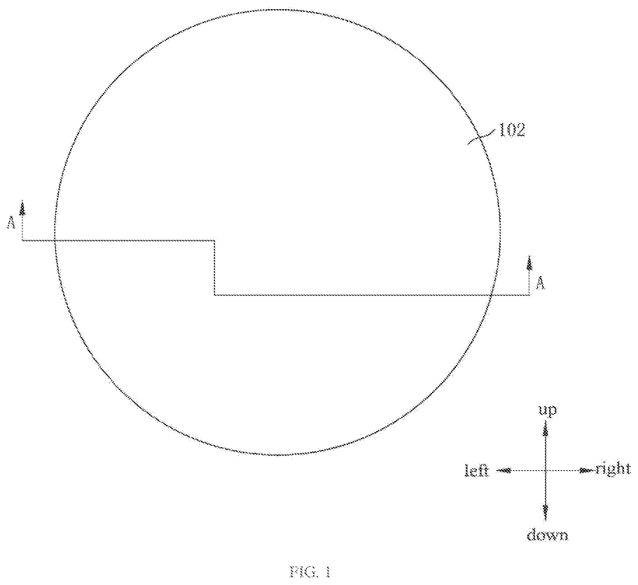

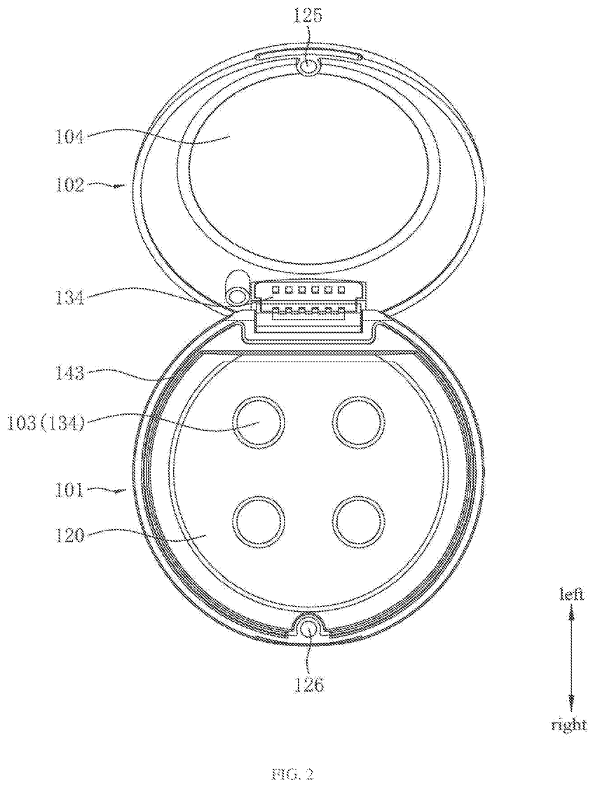

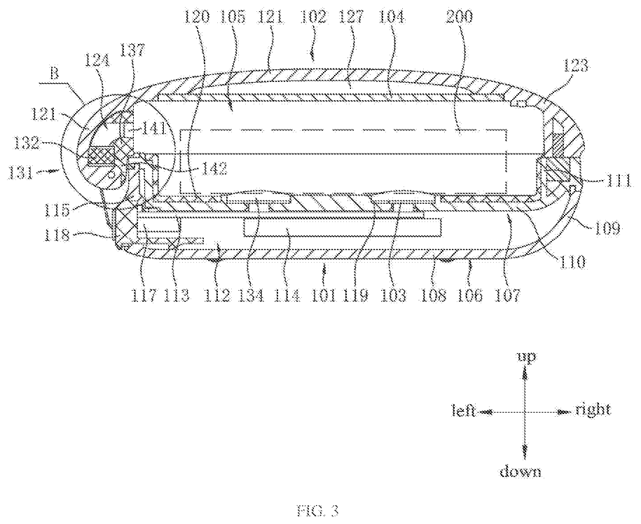

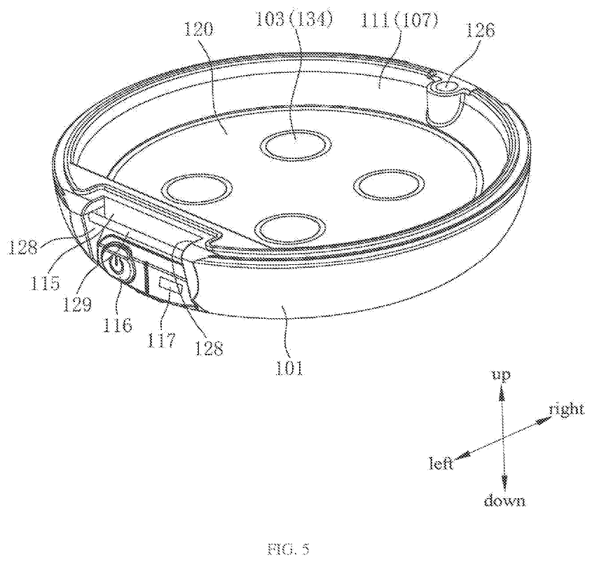

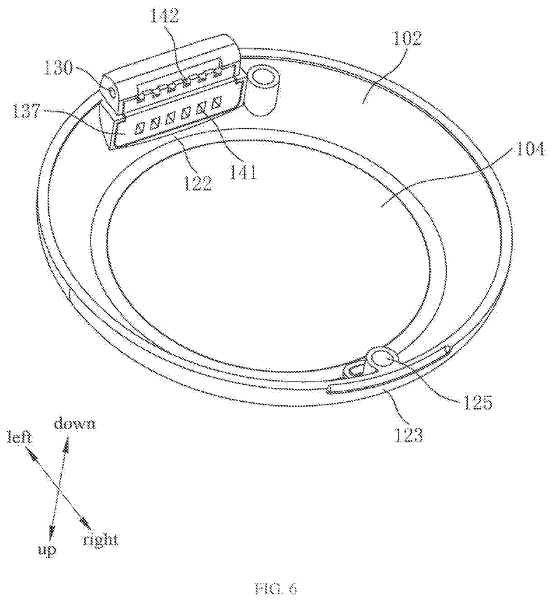

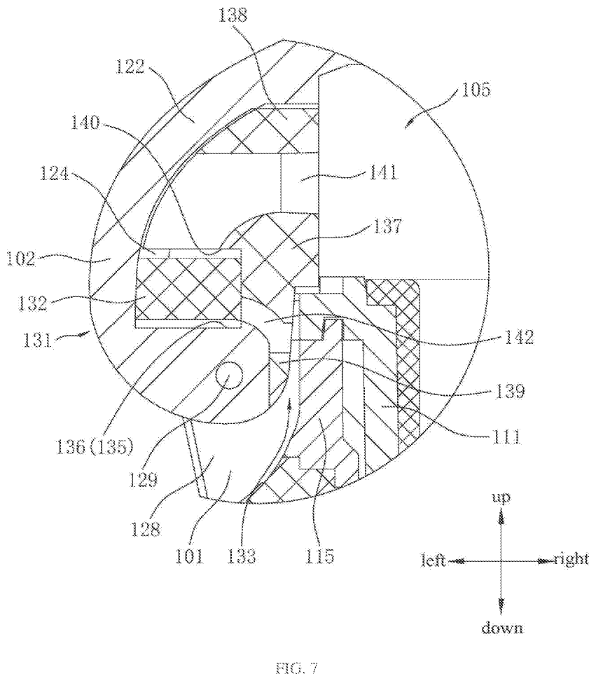

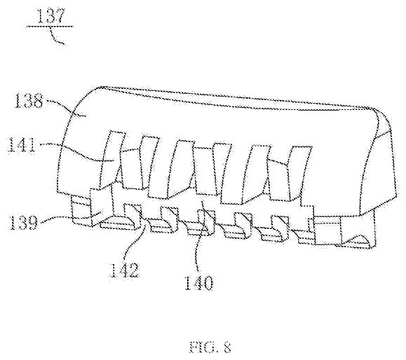

FIG. 1 is a top view of a sterilizing device. FIG. 2 is a schematic diagram of the sterilizing device in an opened state. FIG. 3 is a cross-sectional view along an A-A part in FIG. 1. FIG. 3 is a cross-sectional view along an A-A part in FIG. 1. FIG. 5 is a perspective diagram of a housing 101 of the sterilizing device. FIG. 6 is a perspective diagram of a cover 102 of the sterilizing device. FIG. 7 is an enlarged view of a B part in FIG. 3. FIG. 8 is a perspective diagram of a fixing member 137.

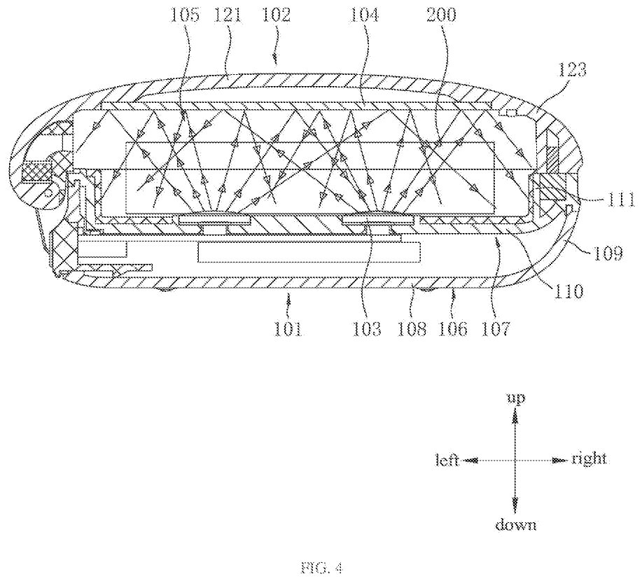

Referring to FIG. 1 to FIG. 8, and mainly referring to FIG. 2, FIG. 3, FIG. 4, FIG. 5 and FIG. 6, a sterilizing device in the present disclosure comprises a housing 101 and a cover 102, a sterilizing lamp 103, and an optical reflector 104. The cover 102 is configured to open or cover relative to the housing 101, and in a state that the cover 102 is configured to cover relative to the housing 101, the housing 101 and the cover 102 jointly form a storage space 105. The sterilizing lamp 103 is mounted on one of the housing 101 or the cover 102, and configured to emit sterilizing light towards the storage space 105. The optical reflector 104 is arranged on the other one of the housing 101 or the cover 102, and opposite to the sterilizing lamp 103 in the storage space 105.

The sterilizing device in the present disclosure is capable of sterilizing an object to be sterilized 200 from more directions (referring to FIG. 4). Specifically, when the object to be sterilized 200, such as a toothbrush, a tableware, a denture and a razor, is placed in the storage space 105 for sterilizing, since the sterilizing lamp 103 mainly emits the sterilizing light towards one surfaces of these personal articles oriented to the sterilizing lamp 103, the other surfaces of these personal articles away from the sterilizing lamp 103 are avoided from the emitted sterilizing light. Referring to FIG. 4, in the embodiment, by the optical reflector 104 arranged in the storage space 105 opposite to the sterilizing lamp 103, the sterilizing lamp 103 emits the sterilizing light towards one surface of the object to be sterilized 200, and then the sterilizing light is reflected by the optical reflector 104 to the other surface of the object to be sterilized 200. Therefore, the object to be sterilized 200 is capable of being sterilized from more directions.

Referring to FIG. 3 and secondarily referring to FIG. 2, the housing 101 and the cover 102 of the sterilizing device in the embodiment are integrally formed into a box shape in a covered state. The box shape may be, for example, a square, a circle, an ellipse, a polygon with more than five sides, and other shapes approximately from a top-view perspective (an up-down direction in the drawing). In order to improve a sterilizing effect of the storage space 105 of the sterilizing device, a sealing ring 143 may be arranged on a periphery of an end face of one of the housing 101 or the cover 102. For example, in FIG. 3, the sealing ring 143 is arranged around the periphery of the end face of the housing 101 opposite to the cover 102. By arranging the sealing ring 143, an airtightness in the storage space 105 in the state that the cover 102 is configured to cover on the housing 101 is improved, so as to improve a sterilizing effect, and in addition, external dust and the like can also be prevented from entering the storage space 105.

Referring to FIG. 3, the housing 101 comprises an outer housing 106 and an inner housing 107, the outer housing 106 is provided with a first substrate 108 and a first annular wall 109 protruding upwardly around a circumference of the first substrate 108, and the inner housing 107 is provided with a second substrate 110 and a second annular wall 111 protruding upwardly around a circumference of the second substrate 110. The inner housing 107 is mounted in the outer housing 106 from above. Mounting modes of the inner housing 107 and the outer housing 106 are not particularly limited, for example, various modes comprising clamping, screwing and bonding may be selected. For example, in the embodiment, the second annular wall 111 of the inner housing 107 is formed by being clamped on the first annular wall 109 of the outer housing 106. In addition, a connection position between the inner housing 107 and the outer housing 106 may be sealed by a sealing ring or a sealant. A control box 112 is formed between the outer housing 106 and the inner housing 107. Electrical components such as a control panel 113 and a battery 114 are stored in the control box 112. A first mounting wall 115 recessing inwardly is formed on the first annular wall 109 of the outer housing 106, and the first mounting wall 115 is in a flat plate shape as a whole. A power switch 116, a charging interface 117 and the like of the sterilizing device are mounted on the first mounting wall 115. In addition, the sterilizing device may also be provided with a sealing plug 118 capable of being clamped on the first mounting wall 115.

Referring to FIG. 3 and FIG. 5, the sterilizing lamp 103 may be mounted on a bottom portion in the housing 101. Specifically, the second substrate 110 of the inner housing 107 of the housing 101 may be provided with a mounting hole 119 for mounting the sterilizing lamp 103. The mounting hole 119 may penetrate through the second substrate 110 of the inner housing 107 in an up-down direction, and is communicated with the control box 112. The sterilizing lamp 103, for example, may be a well-known ultraviolet sterilizing lamp. A light emitting part of the sterilizing lamp 103 may be exposed from the mounting hole 119, and a wiring part of the sterilizing lamp 103 is connected with the control panel 113, for example, in the control box 112. In addition, in order to improve an airtightness of the control box 112, the sterilizing lamp 103 may be hermetically mounted in the mounting hole 119 by a sealing ring or a sealant.

A number of the sterilizing lamps 103 is not particularly limited, and one or more sterilizing lamps (such as more than four sterilizing lamps) may be provided. From a viewpoint of sterilizing the object to be sterilized 200 from more directions, a plurality of sterilizing lamps 103 may be provided, and the plurality of sterilizing lamps 103 are distributed on the bottom portion in the housing 101 at intervals. For example, the sterilizing lamps 103 may be uniformly distributed in a rectangular array or a circumferential array with a center of the second substrate 110 of the inner housing 107 as a reference. Therefore, the sterilizing lamps 103 are capable of being arranged in a plurality of areas in the storage space 105, so that the sterilizing light is capable of being emitted to more areas in the storage space 105 of the sterilizing device.

Referring to FIG. 3 and FIG. 4, in addition, the sterilizing lamp may emit the sterilizing light towards the storage space through an optical refractor 134. Specifically, for example, the light emitting part of the sterilizing lamp 103 is accommodated in the mounting hole 119. The optical refractor 134, such as a plano-concave lens, a plano-convex lens, a convex lens or a concave lens, is mounted in the mounting hole 119. Therefore, by emitting the sterilizing light towards the storage space 105 through the optical refractor 134, the sterilizing light is capable of being emitted into the storage space at various angles relative to an up-down direction, so that the sterilizing light is capable of being emitted towards more areas in the storage space 105 of the sterilizing device. In addition, after the sterilizing light is reflected by the optical reflector 104, an emitting path of the reflected sterilizing light is capable of being further diffused, so as to emit the sterilizing light to an interior of the whole storage space 105, and improve a sterilizing effect.

In a state that the object to be sterilized 200 is stored, the object to be sterilized 200, for example, is placed on an upper side of the second substrate 110 of the inner housing 107. Specifically, the second substrate 110 and the second annular wall 111 of the inner housing 107 integrally form a part of the storage space 105. A height of the second annular wall 111 may be larger than that of the object to be sterilized 200, or lower than that of the object to be sterilized 200. In addition, a gasket 120 may be laid above the inner housing 107. A material of the gasket 120 is not particularly limited, for example, the gasket may be made of a silica gel material. The gasket 120 may be in a shape having a size approximately the same as that of the second substrate 110, and the gasket 120 is directly laid on an upper side of the second substrate 110. In addition, the gasket 120 may have a part laid on the upper side of the second substrate 110, and a part attached to an inner surface of the second annular wall 111. Therefore, by laying the gasket 120, not only the object to be sterilized 200 can be buffered, but also the storage space 105 can be cleaned conveniently, for example, the gasket 120 is capable of being directly disassembled and cleaned.

Referring to FIG. 3 and FIG. 6, the cover 102 is in a circular shape from a top-view perspective. In addition, from a cross-sectional perspective of the cover 102, the cover 102 is in an arc shape. Specifically, the cover 102 is provided with a first arc-shaped portion 121 located in a middle portion in a left-right direction, a second arc-shaped portion 122 located on a left side of the first arc-shaped portion 121, and a third arc-shaped portion 123 located on a right side of the first arc-shaped portion 121. The first arc-shaped portion 121 is arched upwardly, and in the state that the cover 102 is configured to cover on the housing 101, the first arc-shaped portion 121 is arranged opposite to the second substrate 110 of the inner housing 107, thus defining a height of the storage space 105 in an up-down direction. By arching the first arc-shaped portion 121 upwardly, the height of the storage space 105 in the up-down direction can be increased, thus expanding the storage space 105. One end of the second arc-shaped portion 122 is connected with the first arc-shaped portion 121. In addition, the second arc-shaped portion 122 extends downwardly and protrudes outwardly, and is formed as a part protruding outwardly relative to the second annular wall 111 of the inner housing 107 in the state that the cover 102 is configured to cover on the housing 101, and an inner side of this part is used as a part (a channel portion 124, which will be described hereinafter) for air exchange between the storage space 105 and the exterior of the sterilizing device. The third arc-shaped portion 123 is connected with the first arc-shaped portion 121 on one side opposite to the second arc-shaped portion 122. In addition, the third arc-shaped portion 123 extends downwardly, and in the state that the cover 102 is configured to cover on the housing 101, the third arc-shaped portion 123 is approximately attached to the second annular wall 111 of the inner housing 107. A first mounting structure 125 may be arranged at a position on the third arc-shaped portion 123, and correspondingly, a second mounting structure 126 may be arranged at a position on the second annular wall 111 of the inner housing 107. In the state that the cover 102 is configured to cover on the housing 101, the first mounting structure 125 and the second mounting structure 126 are mounted with each other, so that the cover 102 and the housing 101 are mounted with each other. The first mounting structure 125 and the second mounting structure 126 are not particularly limited. For example, the first mounting structure 125 and the second mounting structure 126 may be magnets, snaps and other structures.

The optical reflector 104, for example, may be a reflecting mirror, and is arranged at a top portion in the cover 102. Specifically, the first arc-shaped portion 121 is provided with, for example, a mounting groove 127 on one side (a lower side in the drawing) opposite to the storage space 105, and the optical reflector 104 is mounted in the mounting groove 127. A mounting mode of the optical reflector 104 is not particularly limited, and may be, for example, bonding, clamping and screwing. In addition, a shape of the optical reflector 104 from a top-view perspective may be determined according to a shape of the storage space 105 from a top-view perspective, and may be, for example, a square, a circle, an ellipse, a polygon with more than four sides. Moreover, in the state that the cover 102 is configured to cover relative to the housing 101, the optical reflector 104 completely shields the plurality of the sterilizing lamps 103 from a top-view perspective. A size of the optical reflector 104 is not particularly limited as long as the optical reflector completely shields the plurality of the sterilizing lamps 103 from a top-view perspective. For example, the size of the optical reflector 104 may be larger than a size of the storage space 105, or may be equal to or slightly smaller than the size of the storage space 105. Therefore, an area covered by the optical reflector 104 can be increased as much as possible.

Examples of the reflecting mirror used as the optical reflector 104 comprise a planar reflecting mirror, a convex reflecting mirror and a concave reflecting mirror. When the planar reflecting mirror is selected, the planar reflecting mirror may be mounted in the mounting groove 127 substantially parallel to the second substrate 110 of the inner housing 107. Therefore, the sterilizing light emitted from the sterilizing lamp 103 can be fully reflected. When the convex reflecting mirror or the concave reflecting mirror is selected, the convex reflecting mirror or the concave reflecting mirror may be also be mounted in the mounting groove 127 with an axis substantially coincident with a center of the storage space 105. By selecting the convex reflecting mirror or the concave reflecting mirror, not only the sterilizing light emitted from the sterilizing lamp 103 can be fully reflected, but also the reflected sterilizing light may also be inclined relative to the sterilizing light emitted from the sterilizing lamp 103, so that the object to be sterilized 200 can be sterilized from more directions.

In addition, a plurality of optical reflectors 104 may also be provided, and these optical reflectors 104 may all be the planar reflecting mirrors, the convex reflecting mirrors or the concave reflecting mirrors. In addition, the plurality of optical reflectors 104 may also comprise a combination of the planar reflecting mirror, the convex reflecting mirror and the concave reflecting mirror.

Further, the optical reflectors 104 may also be, for example, optical reflecting films, and these optical reflecting films, for example, may be directly covered on one side (inner side) of the cover 102 opposite to the storage space 105. For example, the optical reflecting films may be covered on the first arc-shaped portion 121, the second arc-shaped portion 122 and the third arc-shaped portion 123. Alternatively, the optical reflectors 104 may be integrally formed with the cover 102. For example, materials such as mirror stainless steel capable of reflecting the sterilizing light such as an ultraviolet ray may be selected, and in this case, a whole inside part in the cover 102 opposite to the storage space 105 may be used as an optical emitter.

In addition, an inner side of the second annular wall 111 of the inner housing 107 of the housing 101 may also be provided with the optical reflector 104. The second annular wall 111 may be partially provided with the optical reflector 104, or may be circumferentially provided with the optical reflector 104. Similarly, the optical reflector 104 may also be the optical reflecting film, or the optical reflector 104 may also be integrally formed with the housing 101.

In addition, it should be noted that the control box 112 and the sterilizing lamp 103 may also be arranged on the cover 102, and in this case, the optical reflector 104 may be arranged on the housing 101.

Alternatively, the control box 112 and the optical reflector 104 are mounted on the housing 101, and the sterilizing lamp 103 is mounted on the cover 102.

Referring to FIG. 3, FIG. 5 and FIG. 6, the cover 102 may be connected with the housing 101 by hinging, clamping, and other ways. From a point of view of facilitating opening and covering the cover 102, the cover 102 may be connected with the housing 101 by hinging. Specifically, as mentioned above, the first mounting wall 115 recessing inwardly is formed on the second annular wall 111 of the outer housing 106. In addition, two second mounting walls 128 are formed on two sides of the first mounting wall 115, and a hinge shaft 129 is erected on the two second mounting walls 128. A tail end of the second arc-shaped portion 122 of the cover 102 is provided with a hinge hole 130, and the cover 102 is hinged with the hinge shaft 129 through the hinge hole 130, so as to be hinged with the housing 101.

Referring to FIG. 7 and FIG. 3, and secondarily referring to FIG. 3 and FIG. 5, since sterilization is carried out in the storage space 105, especially the sterilizing device in the embodiment is additionally provided with the optical reflector 104, the optical reflector 104 greatly increases an area covered by the sterilizing lamp 103 in the storage space 105, and in this case, a temperature in the storage space 105 is increased rapidly, which causes air expansion in the storage space 105. Therefore, the sterilizing device in the embodiment may also be provided with a channel portion 124, and the channel portion 124 communicates the storage space 105 with an exterior of the sterilizing device. By providing the channel portion 124, when air in the storage space 105 expands due to sterilization, the air can be discharged to the exterior through the channel portion 124.

The channel portion 124 is not particularly limited as long as the channel portion can communicate the storage space 105 with the exterior, for example, the channel portion 124 may be formed at a hinged area 131 of the cover 102 and the housing 101, which means that the channel portion 124 may be formed above the first mounting wall 115. In this case, the channel portion 124 may not only realize communication of the storage space 105 with the exterior, but also have a hidden mounting position, thus avoiding affecting an overall appearance of the sterilizing device.

Certainly, the channel portion 124 may also be formed by opening, for example, a ventilation micropore in the cover 102 when necessary.

In addition, after the sterilization is completed, the temperature in the storage space 105 is decreased, and in this case, air is sucked into the storage space 105 again from the exterior through the channel portion 124. In this case, for example, external dust and the like in the exterior may be sucked into the storage space 105 through the channel portion 124, thus causing secondary pollution to the object to be sterilized 200. Therefore, the sterilizing device in the embodiment may be provided with a detachable filtering member 132 in the channel portion 124. A type of the filtering member 132 is not particularly limited, for example, the filtering member may be a filter screen, filter cotton, and the like.

In order to improve a filtration capacity of the filtering member 132, an area, a thickness and the like of the filtering member 132 can be increased. In the sterilizing device in the embodiment, in order to increase the area and/or the thickness of the filtering member 132, it is necessary to increase an area and/or an extension track of the channel portion 124. Therefore, the channel portion 124, for example, may be arranged at the hinged area 131 of the cover 102 and the housing 101. Specifically, as mentioned above, the second arc-shaped portion 122 of the cover 102 extends downwardly and protrudes outwardly, and is formed as a part protruding outwardly relative to the second annular wall 111 of the inner housing 107 (in addition, the part also protrudes outwardly relative to the first mounting wall 115) in the state that the cover 102 is configured to cover on the housing 101, and an inner side of this part is used as the channel portion 124 for communicating the storage space 105 with the exterior of the sterilizing device.

A gap portion 133 for communicating the channel portion 124 with the exterior of the sterilizing device is arranged between the second arc-shaped portion 122 and the first mounting wall 115 of the housing 101, and the gap portion 133 spans the second mounting walls 128 located on two sides of the first mounting wall 115. The channel portion 124 extends from the gap portion 133 into the storage space 105. A supporting surface 136 for supporting the filtering member 132 is formed on an inner wall 135 of the channel portion 124, and the filtering member 132 is supported on the supporting surface 136 and blocks the gap portion 133. Therefore, air is filtered by the filtering member 132 first before entering the channel portion 124 through the gap portion 133, so that dust and the like in the air can be filtered out, thus preventing the dust from entering the storage space 105 through the channel portion 124.

In addition, since the hinged area 131 is arranged at an upper portion of the first mounting wall 115 in a flat plate shape approximately, and the channel portion 124 is arranged at the hinged area 131 of the cover 102 and the housing 101, a cross-sectional length of the channel portion 124 can be greatly increased (which means that the channel portion 124 may span the first mounting wall 115), so that a length of the filtering member 132 accommodated in the channel portion 124 can be increased, thereby enhancing a filtration capacity of the filtering member 132 while enhancing a ventilation capacity of the channel portion 124.

Further, since the second arc-shaped portion 122 of the cover 102 protrudes outwardly and the cover 102 is arched upwardly as a whole, a cross-sectional width of the channel portion 124 and the extension track of the channel portion 124 can be greatly increased, so that a width of the filtering member 132 accommodated in the channel portion 124 and a thickness of the filtering member 132 accommodated in the channel portion 124 can be increased, thereby further enhancing the filtration capacity of the filtering member 132.

Referring to FIG. 7 and FIG. 8, and secondarily referring to FIG. 3 and FIG. 6, in order to reliably fix the filtering member 132 and prevent the filtering member 132 from falling off due to rotation of the cover 102 relative to the housing 101, a detachable fixing member 137 is arranged in the channel portion 124, and the filtering member 132 is fixed in the channel portion 124 by the fixing member 137. A material of the fixing member 137 is not particularly limited, and for example, may be silica gel, rubber and other materials with certain elasticity, so that the fixing member can be easily clamped in the channel portion 124 or detached from the channel portion 124. In addition, the material of the fixing member 137 may also be, for example, a material with an electrostatic adsorption function, thus being able to assist in dust adsorption.

In some embodiments, the fixing member 137 is provided with an upper end portion 138 and a lower end portion 139 integrally formed with the upper end portion 138. A thickness of the upper end portion 138 in a left-right direction is larger than that of the lower end portion 139, which means that the fixing member 137 is approximately formed in a “7” shape as a whole, thus forming an avoidance portion 140 (which will be described hereinafter). The upper end portion 138 of the fixing member 137 is clamped in a space formed by the second arc-shaped portion 122 of the cover 102, the lower end portion 139 of the fixing member 137 extends into the gap portion 133, and a left side of the lower end portion 139 of the fixing member 137 is attached to the inner wall 135 of the channel portion 124. The fixing member 137 may be provided with a ventilation hole 141, a ventilation groove 142, and an avoidance portion 140. The ventilation hole 141 is arranged at one end (the upper end portion 138) of the fixing member 137, and in a state that the fixing member 137 is arranged in the channel portion 124, the ventilation hole 141 is connected with the storage space 105. A plurality of ventilation holes 141 may be provided, and when the fixing member 137 is clamped in the channel portion 124 and the cover 102 is configured to cover relative to the housing 101, the ventilation holes 141 are approximately parallel to the second substrate 110 of the inner housing 107. The ventilation groove 142 is arranged at the other end (the lower end portion 139) of the fixing member 137, and in the state that the fixing member 137 is arranged in the channel portion 124, the ventilation groove 142 is arranged opposite to the inner wall 135 of the channel portion 124 and connected with the exterior of the sterilizing device, which means that the ventilation groove 142 communicates the exterior of the sterilizing device with the channel portion 124. When the fixing member 137 is clamped in the channel portion 124 and the cover 102 is configured to cover relative to the housing 101, the ventilation groove 142 is inclined relative to the ventilation hole 141. The avoidance portion 140 is arranged between the ventilation hole 141 and the ventilation groove 142 in a staggered manner in a left-right direction, and in the state that the fixing member 137 is arranged in the channel portion 124, the avoidance portion 140 is arranged opposite to the inner wall 135 of the channel portion 124 and forms a mounting space for mounting the filtering member 132 together with the inner wall 135. Specifically, since a thickness of the upper end portion 138 in a left-right direction is greater than that of the lower end portion 139, the avoidance portion 140 is formed between a lower surface of the upper end portion 138 and a left side surface of the lower end portion 139, and the mounting space is formed between the inner wall 135 of the channel portion 124 and the avoidance portion 140. Moreover, the mounting space is arranged opposite to the ventilation hole 141 and the ventilation groove 142 in a staggered manner in a left-right direction, so that a height of the mounting space can be increased.

In the embodiment, by detachably mounting the fixing member 137 in the channel portion 124 to form the mounting space for mounting the filtering member 132 together with the channel portion 124, the filtering member 132 can be reliably fixed.

In addition, in the embodiment, by increasing the height of the mounting space, the thickness of the filtering member 132 mounted in the mounting space can be increased, and the filtration capacity of the filtering member 132 can be enhanced.

Further, in the embodiment, by arranging the mounting space in which the filtering member 132 is mounted between the ventilation groove 142 and the ventilation hole 141, and opposite to the ventilation groove 142 and the ventilation hole 141 in a staggered manner in a left-right direction, the extension track of the channel portion 124 is approximately in a U shape with an opening oriented to a right side, so that the extension track of the channel portion 124 can be lengthened. When air in the exterior enters the storage space 105 through the channel portion 124, dust can also be adsorbed due to the lengthening of the extension track of the channel portion 124, thus further reducing dust entering the storage space 105.

Therefore, according to the sterilizing device in the embodiment, by the optical reflector 104 arranged in the storage space 105 opposite to the sterilizing lamp 103, the sterilizing lamp 103 emits the sterilizing light towards one surface of the object to be sterilized 200, and then the sterilizing light is reflected by the optical reflector to the other surface of the object to be sterilized 200. Therefore, the object to be sterilized 200 is capable of being sterilized from more directions, and personal articles having high requirements for sanitary conditions, such as a toothbrush, tableware, a denture and a razor, are sterilized more thoroughly.

Further, by emitting the sterilizing light towards the storage space 105 through the optical refractor 134, the sterilizing light is capable of being emitted into the storage space at various angles relative to an up-down direction, so that the sterilizing light is capable of being emitted towards more areas in the storage space 105 of the sterilizing device. Moreover, after the sterilizing light is reflected by the optical reflector 104, an emitting path of the reflected sterilizing light is capable of being further diffused, so as to emit the sterilizing light to an interior of the whole storage space 105, and improve a sterilizing effect.

In addition, according to the sterilizing device in the embodiment, since the filtering member 132 is arranged in the channel portion 124 that communicates the exterior with the storage space 105, dust and the like entering the storage space 105 through the channel portion 124 can be greatly reduced when air is sucked into the storage space 105 from the exterior through the channel portion 124, thus preventing secondary pollution to the object to be sterilized 200.

Further, according to the sterilizing device in the embodiment, by forming the channel portion 124 in the cover 102 and the hinged area 131, the channel portion not only has a hidden mounting position, but also avoids affecting an overall appearance of the sterilizing device, and an area of the mounting space in the channel portion 124 can be increased, so that the ventilation capacity of the channel portion 124 and the filtration capacity of the filtering member 132 can be enhanced.

Although the embodiments of the present disclosure have been shown and described, it can be understood by those of ordinary skills in the art that: various changes, modifications, substitutions and variations may be made to these embodiments without departing from the principle and purpose of the present disclosure, and the scope of the present disclosure is defined by the appended claims and their equivalents.

What is claimed is:

1. A sterilizing device, comprising:
a housing and a cover, wherein the cover is configured to open or cover relative to the housing, and in a state that the cover is configured to cover relative to the housing, the housing and the cover jointly form a storage space;
a sterilizing lamp mounted on one of the housing or the cover, and configured to emit a sterilizing light towards the storage space; and
an optical reflector arranged on the other one of the housing or the cover, and opposite to the sterilizing lamp in the storage space.

2. The sterilizing device of claim 1, wherein the sterilizing lamp is mounted at a bottom portion in the housing, and the optical reflector is arranged at a top portion in the cover.

3. The sterilizing device of claim 2, wherein a plurality of sterilizing lamps are provided, and the plurality of sterilizing lamps are distributed at intervals at the bottom portion in the housing; and
in the state that the cover is configured to cover relative to the housing, the optical reflector shields the plurality of the sterilizing lamps from a top-view perspective.

4. The sterilizing device of claim 1, wherein the optical reflector is a reflecting mirror.

5. The sterilizing device of claim 1, wherein the sterilizing lamp emits the sterilizing light towards the storage space through an optical refractor.

6. The sterilizing device of claim 1, wherein the sterilizing device is further provided with a channel portion, and the channel portion is configured to communicate the storage space with an exterior of the sterilizing device.

7. The sterilizing device of claim 6, wherein the cover is hinged with the housing, and the channel portion is formed in a hinged area of the cover and the housing.

8. The sterilizing device of claim 7, wherein a detachable filtering member is arranged in the channel portion.

9. The sterilizing device of claim 8, wherein a detachable fixing member is arranged in the channel portion, and the filtering member is fixed in the channel portion by the fixing member.

10. The sterilizing device of claim 9, wherein the fixing member is provided with:
a ventilation hole arranged at one end of the fixing member, wherein in a state that the fixing member is arranged in the channel portion, the ventilation hole is connected with the storage space;
a ventilation groove arranged at the other end of the fixing member, wherein in the state that the fixing member is arranged in the channel portion, the ventilation groove is connected with the exterior of the sterilizing device; and
an avoidance portion arranged between the ventilation hole and the ventilation groove, wherein in the state that the fixing member is arranged in the channel portion, the avoidance portion is arranged opposite to an inner wall of the channel portion and forms a mounting space for mounting the filtering member together with the inner wall.

11. The sterilizing device of claim 2, wherein the sterilizing lamp emits the sterilizing light towards the storage space through an optical refractor.

12. The sterilizing device of claim 3, wherein the sterilizing lamp emits the sterilizing light towards the storage space through an optical refractor.

13. The sterilizing device of claim 4, wherein the sterilizing lamp emits the sterilizing light towards the storage space through an optical refractor.

14. The sterilizing device of claim 2, wherein the sterilizing device is further provided with a channel portion, and the channel portion is configured to communicate the storage space with an exterior of the sterilizing device.

15. The sterilizing device of claim 3, wherein the sterilizing device is further provided with a channel portion, and the channel portion is configured to communicate the storage space with an exterior of the sterilizing device.

16. The sterilizing device of claim 4, wherein the sterilizing device is further provided with a channel portion, and the channel portion is configured to communicate the storage space with an exterior of the sterilizing device.

17. The sterilizing device of claim 1, wherein a sealing ring is arranged on a periphery of an end face of one of the housing or the cover.

18. The sterilizing device of claim 2, wherein the optical reflector comprises an optical reflecting film covered on one side of the cover opposite to the storage space.

19. The sterilizing device of claim 2, wherein the optical reflector is integrally formed with the cover, and material of the cover comprises a material reflecting the sterilizing light.
